(19) United States
(12) Patent Application Publication
(10) Pub. No.: US 2004/0225301 A1
(43) Pub. Date: Nov. 11, 2004

(54) Loop closure apparatus and method

(75) Inventors: John Avi Roop, Crystal, MN (US); Kedar R. Belhe, Minnetonka, MN (US); Catherine Pipenhagen, Chanhassen, MN (US)

Correspondence Address: L. Grant Foster, HOLLAND & HART LLP, P.O. Box 8749, Denver, CO, 80201, US

(73) Assignee: St. Jude Medical, Daig Division, Inc.

(21) Appl. No.: 10/430,661

(22) Filed: May 5, 2003

(51) Int. Cl. A61B 17/04

(52) U.S. Cl. 606/148

(57) ABSTRACT

A suture device and method according to the present invention involves positioning a body of the suture device inside a sheath and extending the body through a vessel wall. The sheath, in one embodiment, is a sheath through which a medical procedure has been performed and which has remained positioned within an opening in the vessel. First and second cannulae, in the form of sharpened needles, are inserted through the vessel wall and through adjacent subcutaneous tissue on opposite sides of the vessel wall opening. A snare or loop is carried by the first needle on a first side of the vessel opening. A suture is carried by the second needle on a second side of the vessel opening. The snare is deployed inside the vessel so that it assumes an opened, pre-oriented, three-dimensional configuration. The suture is then extended into the vessel and positioned inside of the open area formed by the snare or loop. In one embodiment, the snare includes a V-shaped configuration such that as the snare is pulled back into the first needle, the snare closes around and grasps the free end of the suture to hold it securely against the opening in the first needle. After the suture is properly positioned across the vessel opening, the first and second needles are withdrawn away from the vessel wall along with the suture device, and the suture is tied or clamped to approximate tissue surrounding the opening in the vessel.

### FIELD OF THE INVENTION

This invention relates to suturing devices. More specifically, this invention relates to suturing devices for approximating tissue surrounding an opening in a corporeal vessel wall.

### BACKGROUND OF THE INVENTION

Various medical procedures, particularly cardiology procedures, involve accessing a corporeal vessel through a percutaneous sheath. The sheath necessarily requires the formation of a hole or opening in the vessel wall so that a medical procedure can be performed via the sheath. After the particular medical procedure has been performed, the sheath must eventually be removed from the vessel and the access hole in the vessel wall must be closed.

A number of prior vascular closure devices have been developed in attempting to provide a solution for the problem of closing a hole in the vessel wall. Tissue approximation typically involves passing a length of suture into and through adjacent vessel and subcutaneous tissue, across the vessel opening, and back into and through adjacent vessel and subcutaneous tissue. Certain prior closure devices have involved relatively complicated methods and devices for extracting a length of suture from inside the vessel so that the physician can approximate tissue surrounding the hole in the vessel wall through use of the suture.

U.S. Pat. No. 5,643,292 and U.S. Pat. No. 6,059,800 disclose example prior suturing devices used for approximating tissue surrounding the opening in a vessel wall. Most prior closure devices enlarge the vessel opening thereby negating the benefits of using smaller or less invasive percutaneous products. Prior suturing devices are also relatively complicated and difficult to use.

There remains a need, therefore, to provide a suturing apparatus that is relatively simple in construction, is easy to use, and can effectively approximate tissue surrounding an opening in a vessel wall. There is further a need to provide a suturing device that minimizes the invasiveness of the suturing procedure.

### SUMMARY OF THE INVENTION

The present invention involves a novel snare or loop used to capture a free end of a suture within a vessel to approximate tissue surrounding an opening in a corporeal vessel. The novel suturing apparatus involves two cannulae in the form of sharpened needles through which the suturing elements are deployed. A first needle is inserted through the vessel and through adjacent subcutaneous tissue on one side of the opening in the vessel. A second needle is inserted through the vessel and through adjacent subcutaneous tissue on an opposite side of the opening. A snare or loop is carried by the first needle on a first side of the vessel opening. The snare is then deployed and precisely positioned to surround the opening of the second needle. After the snare has been properly positioned inside of the vessel, a suture, carried by the second needle, is extended into the vessel and through an opening formed by the snare or loop. One end of the snare includes a V-shaped configuration such that as the snare is pulled back into the first needle, the loop closes and the V-shaped area grasps the suture and pulls it toward and ultimately against the opening in the first needle. After the suture is properly placed across the vessel opening, the first and second needles are withdrawn from the vessel wall, while the free end of the suture is held against the opening of the first needle and an additional length of suture is allowed to pass through the second needle and across the vessel opening as the needles are withdrawn. With the suture extending across the vessel opening, and with the suture device being removed from the vessel opening, the suture can be tied or clamped to approximate tissue surrounding the opening in the vessel.

The foregoing and other features, utilities, and advantages of the invention will be apparent from the following detailed description of the invention with reference to the accompanying drawings.

### BRIEF DESCRIPTION OF THE DRAWINGS

FIG. 1 is a perspective view, partly in section, of a suture device shown extending through an opening in a vessel wall, the suture device being deployed via a sheath through which a medical device may have been inserted for performing a medical procedure;

FIG. 2 is a perspective view, partly in section, of the suture device of FIG. 1 with feet being deployed to allow tactile feedback to the person performing the procedure so that the location of the device relative to the vessel wall can be determined;

FIG. 3 is a perspective view, partly in section, of a pair of needles inserted through the vessel wall on opposite sides of the suture device of FIG. 1;

FIG. 4 is a perspective view, partly in section, of the suture device as shown in FIG. 3 with a snare extending from the first needle and positioned to surround the opening of the second needle;

FIG. 5 is a perspective view, partly in section, of the suture device of FIG. 3, showing a suture extending from the second needle and into the vessel through the area encompassed by the snare; and

FIG. 6 is a perspective view, partly in section, of the suture device of FIG. 3, showing the suture captured by the snare and pulled against the opening of the first needle.

### DETAILED DESCRIPTION OF THE INVENTION

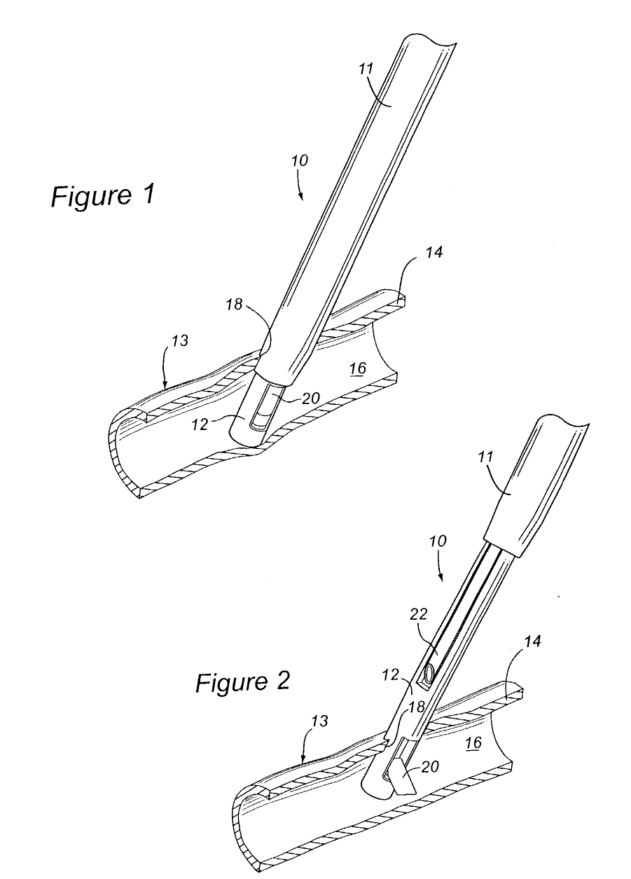

The present invention involves a suture or closure device 10 (FIGS. 1-6) for approximating the tissue surrounding an opening in a corporeal vessel wall after a medical procedure has been performed through such opening. Any type of medical procedure may be performed through a sheath 11 inserted through the opening in the vessel wall to access the inside of the vessel. The closure device 10 utilizes the same sheath 11 for employing the suture to close the opening in the vessel wall.

FIGS. 1 and 2 show the body 12 of the suture or closure device 10 extending beyond a sheath 11 through an opening 18 in a vessel wall 14 to access the interior or inside 16 of the vessel 13. The suture device is deployed via the sheath 11, now retracted away from opening 18. The sheath 11, in one embodiment, is the same sheath through which a medical procedure has been performed. By using the same sheath 11 for both the medical procedure and the suture procedure, the size of opening 18 can be minimized.

In the embodiment of FIGS. 1 and 2, a pair of feet 20 (only one shown) are deployed to extend outwardly from the body 12 of the suture device 10 so that the suture device 10 can be retracted and the feet will engage the inside surface of the vessel wall and provide tactile feedback to the user of the device 10. Thus, the depth of the suture device 10 relative to the vessel wall 14 can be determined by such tactile feedback.

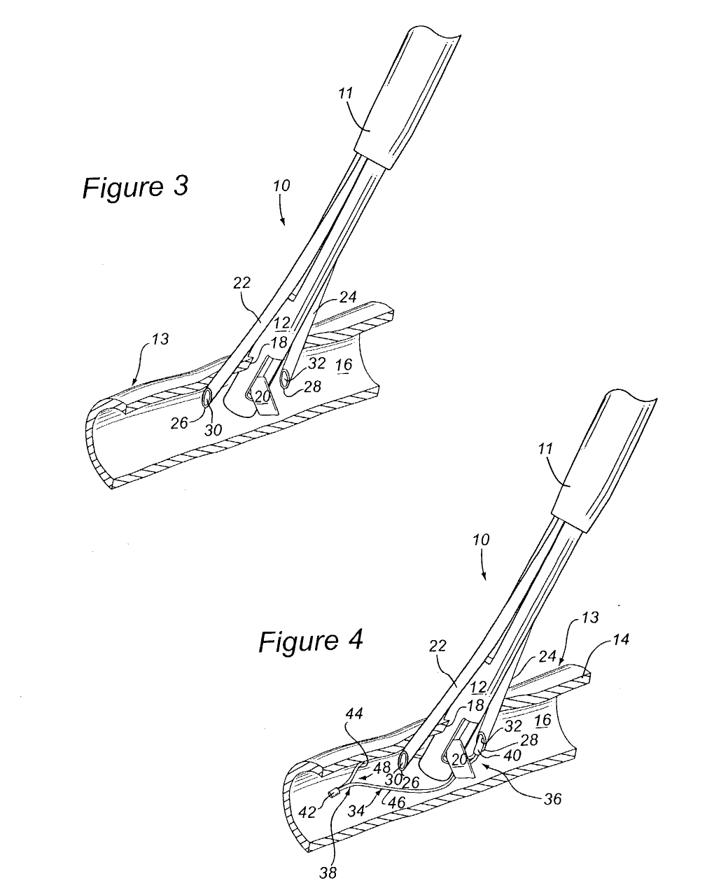

As shown in FIGS. 3-6, a pair of cannulae in the form of sharpened needles 22, 24 are inserted through the subcutaneous tissue (not shown) and the vessel wall 14, so that their respective distal ends 26, 28 extend into the inside 16 of the vessel 13. A first cannula or needle 24 forms a first aperture on one side of the vessel opening, and a second cannula or needle 22 forms a second aperture on an opposite side of the vessel opening. The needles 22, 24 provide passageways 30, 32, respectively, for carrying or allowing insertion of certain suture implements (discussed below). The needles 22, 24 can form an integral part of suture device body 12 such that when the feet 20 are deployed and the depth of the suture device body 12 relative to the vessel wall 14 is determined, precision depth insertion of needles 22, 24 through the vessel wall can be accomplished to ensure that the distal ends 26, 28 are properly positioned beyond the vessel wall 14 on opposite sides of the vessel opening 18. Preferably, the needles 22, 24 will extend at an angle away from the suture device body 12. An angle of 3° to 20° may be utilized, with an angle range of 5° to 6° being preferred.

After the needles 22, 24 have been deployed and extend through the vessel wall 14, a snare 34 in the form of a wire loop, carried by the needle 24, is extended beyond the distal end 28 of needle 24. As shown in FIG. 4, the snare 34, after it has been completely extended past the opening 32 of needle 24, assumes a three-dimensional loop configuration having a proximal end 36 and a distal end 38. The proximal end 36 is formed by two wires 44, 46 held together by a tube 40. The tube 40 securing the wires 44, 46 to form the loop is sized so that it can move through the passageway provided by needle 24. The distal end 38 of loop 34 is held together by a band 42 that is welded, crimped, or otherwise secured to the ends of wires 44, 46. The band 42 is also sized so that it can move inside the passageway formed by needle 24.

As discussed, the loop is formed of two wire members 44, 46. The wires 44, 46 may be made of a nickel/titanium alloy and will preferably have shape memory characteristics such that the configuration of the wire loop 34 is predictable and repeatable. Those skilled in the art will understand that other materials may be used to form the loop 34 without departing from the scope and spirit of the present invention.

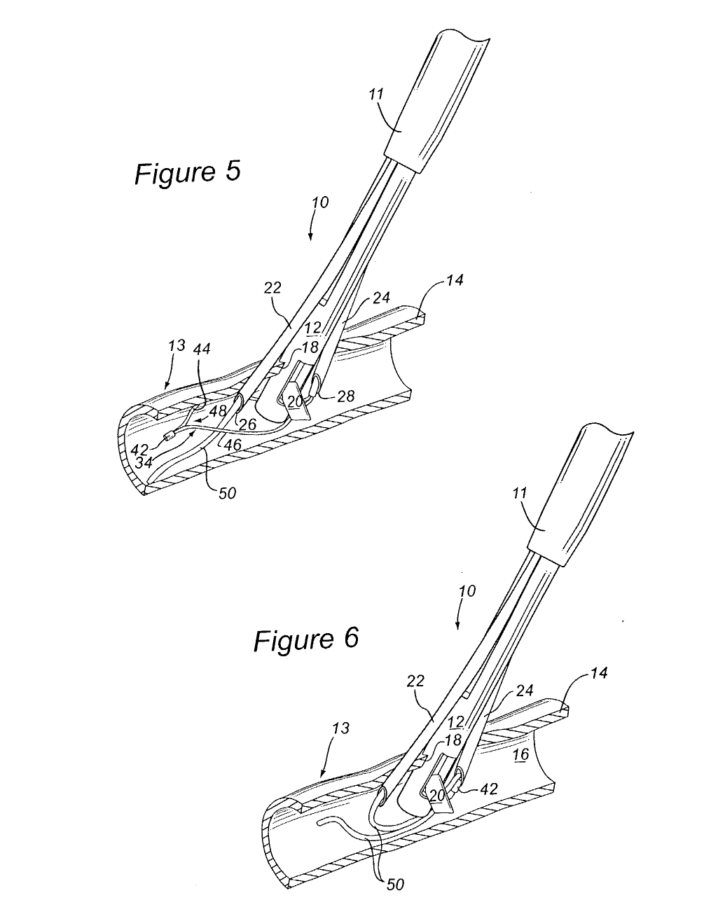

When the snare or wire loop 34 is pulled inside of the first needle 24, wires 44, 46 are pulled together and are held in a side-by-side manner within the needle 24. When the wires 44, 46 are pushed completely outside of the opening 32 of needle 24, the band 42 first moves along the bottom of the inside 16 of the vessel, after which the wire flips up to surround the opening 30 of needle 22. This “flipping” or pivotal movement of the loop 34 prevents interference with the suture device body 12 and the needle 22. When the loop 34 is deployed and wires 44, 46 are spread away from each other, the wires 44, 46 take on a three-dimensional shape (as compared to the two-dimensional, side-by-side, shape the wires assume when inside of needle 24). When the loop is deployed inside the vessel, the distal end 38 of wire loop 34 assumes a generally V-shaped configuration 48 (FIGS. 4 and 5). When an end of suture 50 (FIG. 5) is inserted through second needle 22, the suture 50 extends through the area defined by the snare or wire loop 34. The V-shaped end 48 of the wire loop 34 is configured such that when the wire loop 34 is retracted or pulled back into opening 34 of needle 24, the wire loop 34 closes and the V-shaped area 48 restricts around suture 50 to grasp or capture the suture and allow the suture 50 to be pulled firmly against the opening 34 of first needle 24, as shown in FIG. 6. After the suture 50 has been captured, and has been pulled against the opening of needle 24, the entire apparatus, including the first needle 22, the suture device body 12 (after legs 20 are retracted), and the second needle 24 are retracted together as a unit from the vessel and subcutaneous tissue. An additional length of the suture 50 extending from needle 22 is allowed to pass freely and relatively friction-free through the passageway formed by needle 22 when the entire suture device 10 is removed from the vessel opening 18. As a result, the suture 50 remains in a position spanning the opening 18 and extending through the vessel 13 on opposite sides of the opening 18. The ends of suture 50 can then be drawn together, through a conventional knot or clamp, to close the opening 18 in the vessel wall 14.

In operation, the sheath 11 is inserted into an opening 18 in the vessel 13 to perform a medical procedure. After the medical procedure has been accomplished, the suture device 10 is inserted through the sheath 11 and through the opening 18. The suture device body 12 will then extend into the vessel interior 16, and the sheath 11 is retracted away from opening 18 to expose needles 22, 24 carried by the suture device body 12. The feet 20 are then deployed outwardly from the suture device body 12. The suture device body 12 is then pulled upwardly so that tactile feedback indicates to the person utilizing the device 10 that the proper depth into vessel 13 has been achieved. Thereafter, cannulae in the form of sharpened needles 22, 24 are inserted through the subcutaneous tissue (not shown) and the vessel wall 14 such that they extend into the inside 16 of the vessel 13 at a known depth. A snare, in the form of a wire loop 34, is carried by the first needle 24 and is extended beyond the opening 32 of needle 24 so that the loop 34 passes into the interior 16 of vessel 13. When deployed, the snare 34 assumes the configuration of a three-dimensional wire loop. The wire loop 34 first moves along the bottom of the interior 16 of vessel 13, and then pivots or flips over when deployed to avoid contact with the suture device body 12. The wire loop 34, when deployed, surrounds an aperture 30 of a second needle 22 which carries a suture 50. The suture 50 is then extended beyond the opening 30 in needle 22 (FIG. 5) so that the suture 50 is positioned within the interior of the open area encompassed by wire loop 34. As the wire loop 34 is pulled back to the inside of needle 24, the loop 34 closes around the suture 50 such that the V-shaped area 48 clamps or grasps the suture 50 so that the snare 34 and the suture 50 can be pulled against the opening of first needle 24 (FIG. 6). After suture 50 has been pulled firmly against opening 32 of needle 24, the legs 20 are retracted and the entire assembly 10, including the first needle 24, the suture device body 12, and the second needle 22, is retracted from the vessel and adjacent subcutaneous tissue so that the suture 50 extends across opening 18 and can be tensioned to approximate tissue surrounding opening 18.

While this invention has been described with reference to certain specific embodiments and examples, it will be recognized by those skilled in the art that many variations are possible without departing from the scope and spirit of this invention. The invention, as defined by the claims, is intended to cover all changes and modifications of the invention which do not depart from the spirit of the invention. The words “including” and “having,” as used in the specification, including the claims, shall have the same meaning as the word “comprising.”

1. A suturing device, comprising:
a first cannula being insertable through a vessel wall on a first side of a vessel opening;
a second cannula being insertable through the vessel wall on a second side of a vessel opening;
a snare positioned inside the first cannula, the snare being extendable beyond the first cannula and into the vessel;
a suture having a free end carried by the second cannula, the suture free end extendable beyond the second cannula and into the vessel;
wherein the snare is collapsible to grasp the free end of the suture and pull the suture across the vessel opening and against the first needle to approximate tissue surrounding the vessel opening.

2. A suturing device according to claim 1 wherein the snare comprises a loop.

3. A suturing device according to claim 1 wherein the snare comprises a V-shaped loop.

4. A suturing device according to claim 1 wherein the snare comprises a wire loop having a memory that causes the wire loop to open in a repeatable orientation.

5. A suturing device according to claim 1 wherein the snare comprises a V-shaped wire loop having a memory that causes the wire loop to open in a repeatable orientation.

6. A suturing device according to claim 1 wherein the snare comprises a V-shaped wire loop having a memory that causes the wire loop to open in a repeatable orientation so that the loop surrounds the second cannula and receives the free end of the suture.

7. A suturing device according to claim 1 wherein the snare comprises a pair of wires each having a distal end, the wires being joined together at their distal ends to form a V-shaped end.

8. A suturing device according to claim 1 wherein the snare comprises a pair of wires each having a distal end, the wires being joined together by a band at their distal ends to form generally a V-shaped end.

9. A suturing device according to claim 1, further comprising a device body positioned inside the vessel opening, wherein the snare comprises a V-shaped wire loop having a memory that causes the wire loop to open in a repeatable orientation so that the loop surrounds the second cannula, avoids interference with the device body, and receives the free end of the suture.

10. A suturing device, comprising:
a snare being insertable into a first aperture formed in a vessel wall on a first side of a vessel opening, the snare being extendable into the vessel;
a suture having a free end, the suture being insertable into a second aperture formed in the vessel wall on a second side of a vessel opening, the free end of the suture being extendable into the vessel;
the snare being positionable to surround the free end of the suture and capture the suture such that the suture can be pulled through the first aperture to approximate tissue surrounding the vessel opening.

11. A suturing device according to claim 10 wherein the snare comprises a loop.

12. A suturing device according to claim 10 wherein the snare comprises a V-shaped loop.

13. A suturing device according to claim 10 wherein the snare comprises a wire loop having a memory that causes the wire loop to open in a repeatable orientation.

14. A suturing device according to claim 10 wherein the snare comprises a V-shaped wire loop having a memory that causes the wire loop to open in a repeatable orientation.

15. A suturing device according to claim 10 wherein the snare comprises a V-shaped wire loop having a memory that causes the wire loop to open in a repeatable orientation so that the loop is positioned below the second aperture to receive the free end of the suture.

16. A suturing device according to claim 10 wherein the snare comprises a pair of wires each having a distal end, the wires being joined together at their distal ends to form generally a V-shaped end.

17. A suturing device according to claim 10 wherein the snare comprises a pair of wires each having a distal end, the wires being joined together by a band at their distal ends to form generally a V-shaped end.

18. A suturing device according to claim 10 further comprising a suture device body positioned inside the vessel opening, wherein the snare comprises a V-shaped wire loop having a memory that causes the wire loop to open in a repeatable orientation so that the loop is positioned below the second aperture, receives the free end of the suture, and does not interfere with the suture device body.

19. A method of suturing a vessel wall, comprising:
providing a snare;
inserting the snare into a first aperture formed in the vessel wall on a first side of a vessel opening;
providing a suture having a free end;
inserting the suture into a second aperture formed in a vessel wall on a second side of a vessel opening;
extending the snare into the vessel;
extending the free end of the suture into the vessel and inside the snare;
capturing the suture with the snare;
pulling the suture through the first aperture to approximate tissue surrounding the vessel opening.

20. The method of claim 19 wherein the snare is inserted through a first cannula positioned on the first side of the vessel opening and the suture is inserted through a second cannula positioned on the second side of the vessel opening.

21. The method of claim 19, further comprising:
inserting the snare through a first cannula positioned on the first side of the vessel opening;
inserting the suture through a second cannula positioned on the second side of the vessel opening;
positioning a suture device body in the vessel opening;
extending the snare into the vessel to avoid contact with the suture device body and to surround the second aperture to receive and capture the suture.

22. The method of claim 19, further comprising:
positioning a suture device body in the vessel opening;
deploying the snare to avoid contact with the suture device body and to surround the second aperture to receive and capture the suture.

23. The method of claim 19 further comprising:
repeatedly orienting the snare in the same orientation as the snare is inserted through the first aperture and into the vessel to surround the second aperture.

24. The method of claim 19 further comprising:
maintaining the snare in a closed position before the snare is inserted through the first aperture;
providing the snare with a memory so that as the snare is inserted into the vessel, the snare assumes a predictable orientation for surrounding the second aperture.

25. A method of suturing a vessel wall, comprising:
providing a loop;
inserting the loop through a first aperture formed in the vessel wall on a first side of a vessel opening;
providing a suture having a free end;
inserting the suture through a second aperture formed in a vessel wall on a second side of a vessel opening;
extending the free end of the suture into the vessel;
extending the loop into the vessel;
positioning the loop to surround the free end of the suture;
constricting the loop to capture the suture within the loop;
drawing the suture and loop through the first aperture in the vessel to approximate tissue surrounding the vessel opening.

26. The method of claim 25 wherein the suture is inserted through a second cannula positioned on the second side of the vessel opening and the loop is inserted through a first cannula positioned on the first side of the vessel opening.

27. The method of claim 25, further comprising:
inserting the loop through a first cannula positioned on the first side of the vessel opening;
inserting the suture through a second cannula positioned on the second side of the vessel opening;
positioning a suture device body in the vessel opening;
extending the loop into the vessel to avoid contact with the suture device body and to surround the second aperture to receive and capture the suture.

28. The method of claim 25, further comprising:
positioning a suture device body in the vessel opening;
deploying the loop to avoid contact with the suture device body and to surround the second aperture to receive and capture the suture.

29. The method of claim 25 further comprising:
repeatedly orienting the loop in the same orientation as the loop is inserted through the first aperture and into the vessel to surround the second aperture.

30. The method of claim 25 further comprising:
maintaining the loop in a closed position before the loop is inserted through the first aperture;
providing the loop with a memory so that as the loop is inserted into the vessel, the loop assumes a predictable orientation for surrounding the second aperture.
